(19) United States
(12) Patent Application Publication
(10) Pub. No.: US 2015/0148723 A1
(43) Pub. Date: May 28, 2015

(54) Decorated Braces, Bandages, and Clips Thereof

(71) Applicant: Tracey Huff, Westwood, NJ (US)

(72) Inventor: Tracey Huff, Westwood, NJ (US)

(21) Appl. No.: 14/553,194

(22) Filed: Nov. 25, 2014

Related U.S. Application Data
(60) Provisional application No. 61/909,523, filed on Nov. 27, 2013.

(51) Int. Cl. A61F 15/00; A61F 5/01

(52) U.S. Cl. CPC A61F 15/00; A61F 5/01
USPC 602/5; 602/60

(57) ABSTRACT

Decorated braces, bandages, and clips thereof are provided herein. More specifically, provided herein is a support brace comprising a fabric material formed in a cylindrical shape to provide support to a body part, the fabric material defining a top opening and a bottom opening, and one or more decorative elements applied to an outer surface of the brace to decorate the brace. Also provided herein is a decorated support bandage set comprising a support bandage strip, one or more decorative elements applied to the support bandage strip over at least a portion of the support bandage strip, and one or more decorated clips, each decorated clip comprising a top having a decorative element, and a bottom having protruding teeth to engage a surface of the decorated bandage wrap to secure an end of the support bandage strip against itself after it is wrapped about a person.

### CROSS-REFERENCE TO RELATED APPLICATIONS

This application claims priority to U.S. Provisional Patent Application No. 61/909,523 filed on Nov. 27, 2013, the entire disclosure of which is expressly incorporated herein by reference.

### BACKGROUND

1. Field of the Disclosure

The present disclosure relates to decorated braces, bandages, and clips thereof. More specifically, the present disclosure relates to decorated knee braces, decorated bandage wraps, and decorated bandage clips.

2. Related Art

Children and adults often have injuries requiring a brace or bandage (e.g., sprains, breaks, fractures, etc.). However, such braces and bandages are plain and often noticeable depending on the attire and type of injury (e.g., a knee brace worn with a dress or skirt). People, especially children, may be particularly self-conscious about the bandage or brace, especially at social gatherings, whether formal or informal (e.g., school, parties, weddings, etc.). This can lead to non-use which can result in further injury or prolonging recovery from injury.

What would be desirable, but has not yet been developed, is a brace or bandage that is more likely to be worn by an injured person.

### SUMMARY

The present disclosure relates to decorated braces, bandages, and clips thereof. More specifically, the present disclosure relates to decorated braces, decorated bandage wraps, and decorated bandage clips. The braces and bandages could be decorated with any of a variety of designs or decorative elements using any suitable technique. The designs could relate to anything that an injured person might want to display such that the bandage or brace is personalized for the injured person. The braces, bandages, and/or bandage clips could be packaged in any of a variety of ways, such as in a decoration kit.

### BRIEF DESCRIPTION OF THE DRAWINGS

The foregoing features of the disclosure will be apparent from the following Detailed Description, taken in connection with the accompanying drawings, in which:

FIG. 1 is a perspective view of a decorated brace;

FIG. 2 is a perspective view of another decorated brace;

FIG. 3 is a perspective view of a decorated bandage;

FIG. 4 is a perspective view of a decorated bandage clip;

FIG. 5 is a front view of the decorated bandage clip of FIG. 4; and

FIG. 6 is a back view of the decorated bandage clip of FIG. 4.

### DETAILED DESCRIPTION

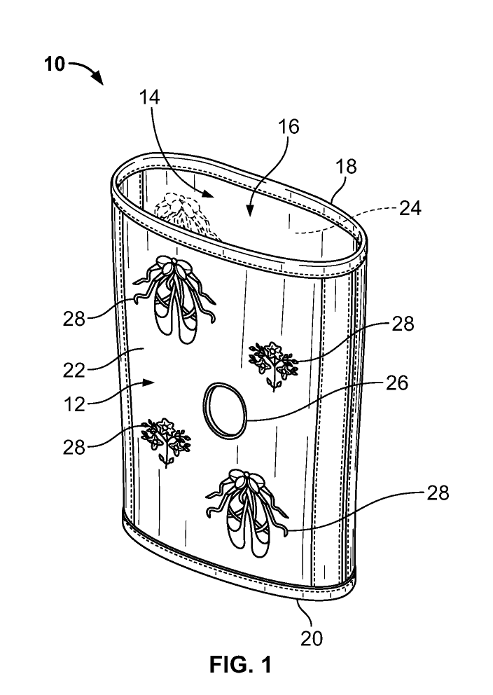

The present disclosure relates to decorated braces, bandages, and clips thereof, as discussed below in connection with FIGS. 1-6.

The braces or bandages (or any other type of support) of the present disclosure could be made of any of a variety of materials, such as fabric (e.g., cotton, silk, wool or a synthetic fabric). The brace could be any type of brace or other support (e.g., knee brace, elbow brace, wrist brace, ankle brace, etc.), and the bandage could be any type of bandage or other support (e.g., Ace bandage wrap (Ace is a registered trademark of 3M Company, St. Paul, Minn.), cold-compress wrap, etc.).

The braces, bandages, and/or clips disclosed herein could be decorated with any of a variety of designs and/or decorative elements using any suitable technique. For example, the decoration could be any design including any one or combination of solid colors, patterns (e.g., stripes, plaid, tie-dye, polka dot, geometric, etc.), and/or objects (e.g., flowers, sports equipment, television characters, movie characters, mythical creatures, etc.), etc. Further, the design could be at any one or multiple locations on the brace, bandage, and/or clip (e.g., cover all or a portion of the decorated knee brace). The design could be a simple design which could cover the entire surface of the brace or bandage. Suitable techniques could include stitching, screen printing, embroidery, painting, dyeing, printing, waxing, burning (e.g., burning an image into the fabric), rusting, digital printing, felting, etc. Materials that could be used to decorate the braces or bandages include heat transfer paints or crayons, paint sticks, decolourant, fabrics (e.g., by layering different types of fabrics), resists (e.g., beeswax, soy wax, water-based resist, etc.), and/or other coloring utensils, etc. Additionally, other objects (e.g., other decorative elements) can be attached (e.g., ironed, sewn, etc.) onto the fabric, including beads, crystals, buttons, charms, fibers, patches, etc. Further, these coloring utensils, materials, etc. can be provided in a packaged set or kit, as described in more detail below.

FIG. 1 is a perspective view of a decorated brace. As shown, the decorated brace 10 includes a fabric material formed in a generally hollow cylindrical shape. The fabric material is usually flexible (e.g., deformable) and stretches to ensure a tight fit to support or prevent particular motion of the injured body part. The brace 10 includes an outer surface 12 and an inner surface 14 defining an interior 16. The brace 10 further includes a top 18 defining an opening (e.g., usually circular), a bottom 20 defining a bottom opening (e.g., usually circular), a front 22, and a back 24. The brace further includes an optional hole 26 through the front 22 (e.g., for a knee or elbow).

The brace 10 has one or more decorative elements 28 applied to the outside surface 12 of the brace 10. In this particular embodiment, the decorative elements 28 include ballet shoes and flowers, however, any type or form of decoration could be used. Accordingly, an injured person could pick a brace from numerous choices bearing a variety of designs. In this way, the injured person can wear a personalized brace: a brace decorated with a design that says something about the injured person, such as the person's favorite sport or sports team, etc. Additionally, the decorative elements 28 could also be on the inside surface 14 so that the brace 10 could be reversible or flipped inside out (e.g., have two different displays of decorative elements).

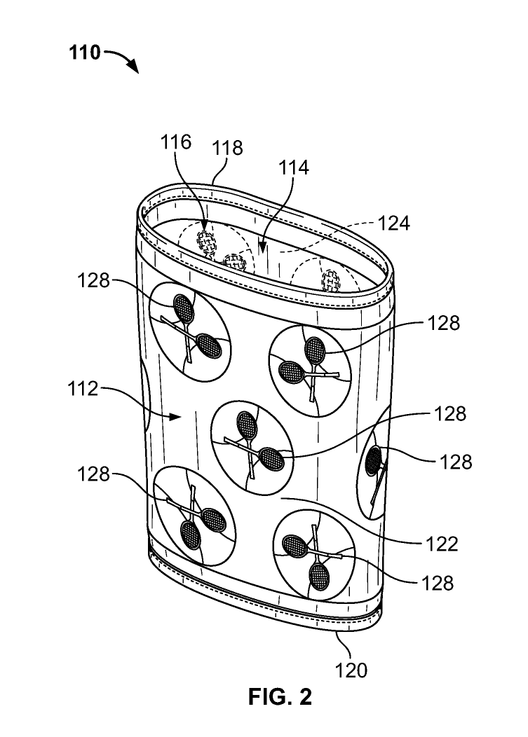

FIG. 2 is a perspective view of another decorated brace 110. As with the brace 10 of FIG. 1, the brace 110 includes an outer surface 112, an inner surface 114 defining an interior 116, a top 118 defining a top opening, a bottom 120 defining a bottom opening, a front 122, and a back 124. The brace 110 has one or more decorative elements 128 applied to the outer surface 112 (and/or inner surface 114) of the brace 110. In this particular embodiment, the decorative elements 128 include tennis designs, each design displaying tennis racquets and a tennis ball. However, any type or form of decoration could be used.

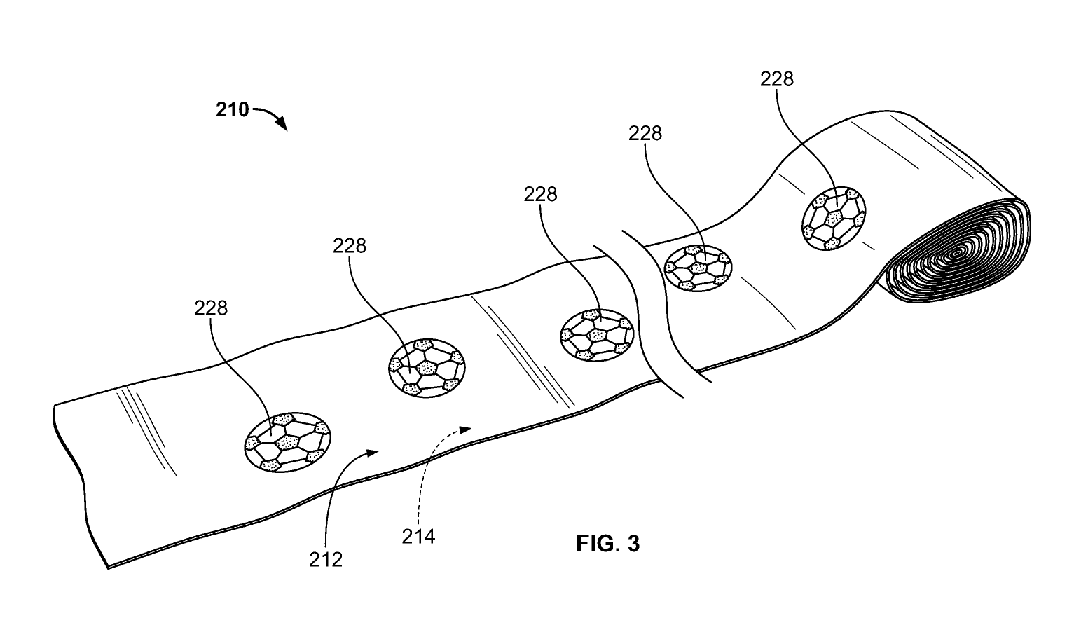

FIG. 3 is a perspective view of a decorated bandage 210. The bandage 210 could be any suitable bandage (e.g., Ace bandage wrap). The bandage 210 has a front surface 212 and a back surface 214. The front surface 212 includes decorative elements 228 that could extend for a portion or the entire length of the bandage 210. The decorative elements 228 include soccer balls, however, any type or form of decoration could be used. The bandage 210 could be cut to any desired length.

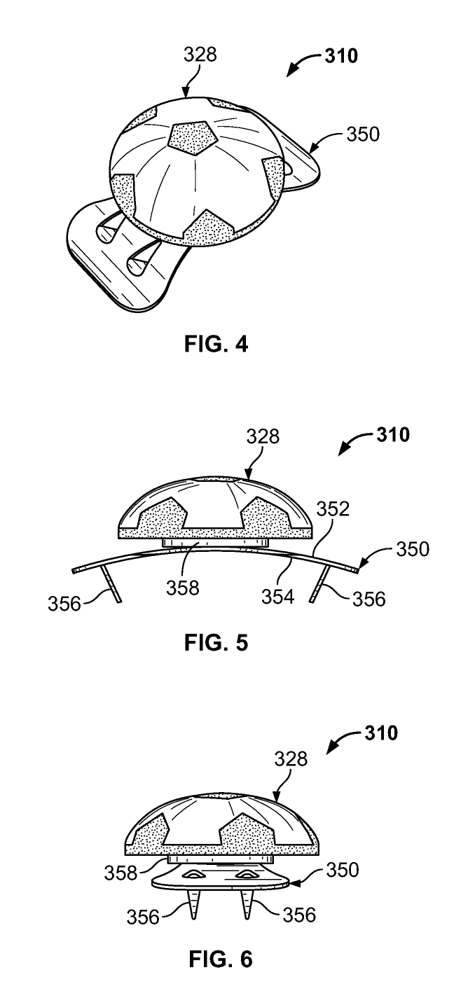

FIGS. 4-6 are views of a decorated bandage clip 310. More specifically, FIG. 4 is a perspective view of a decorated bandage clip 310, FIG. 5 is a front view of the decorated bandage clip 310, and FIG. 6 is a side view of the decorated bandage clip 310. The decorated bandage clip 310 includes a clip 350 having a top 352 and bottom 354, with teeth 356 protruding from the bottom 354. The teeth 356 engage the surface of a bandage to secure the end of the bandage against the bandage so it stays on a user and prevents the bandage from unraveling (e.g., Ace bandage). The decorated bandage clip 310 further includes a decorative element 328 applied to the top 352 of the clip 350, such as via an adhesive 358 (e.g., double-sided tape, glue, hook-and-loop fasteners, etc.). In this particular embodiment, the decorative element 328 is a soccer ball, however, any decorative element could be used. As can be seen, the decorative element can be attached to the clip, but it should be understood that the decorative element could subsume the clip such that the teeth extend from the decorative element. The decorative element 328 could be formed integrally with the clip 310, such that the teeth extend from the decorative element, or the decorative element 328 and the clip 310 could be applied together or attached during the manufacturing process of the bandage clips 310 (e.g., the manufacturing process of the metal bandage clips could include attachment of the decorative elements).

The braces, bandages, and/or bandage clips could be packaged in any of a variety of ways. For example, the decorated bandage of FIG. 5 and decorated bandage clip of FIGS. 4-6 could be packaged separately or packaged together as a set (e.g., wherein the decorative elements of the bandage and clip match or complement one another). Further, the bandage, brace, and/or bandage clip could come as a prepackaged kit for a user to personalize with decorative elements within the kit and/or other decorative elements packaged separately. These decorative elements could include decorative materials (e.g., heat transfer paints or crayons, paint sticks, decolourant, fabrics , resists, and/or other coloring utensils, etc.), and/or other decorative objects (e.g., beads, crystals, buttons, charms, fibers, patches, etc.). Alternatively, or additionally, the brace, bandage, and/or clip could also be partially or fully pre-decorated in any particular fashion.

Having thus described the system and method in detail, it is to be understood that the foregoing description is not intended to limit the spirit or scope thereof. It will be understood that the embodiments of the present disclosure described herein are merely exemplary and that a person skilled in the art may make any variations and modification without departing from the spirit and scope of the disclosure. All such variations and modifications, including those discussed above, are intended to be included within the scope of the disclosure.

What is claimed is:

1. A support brace comprising:
a fabric material formed in a cylindrical shape to provide support to a body part, the fabric material defining a top opening and a bottom opening; and
one or more decorative elements applied to an outer surface of the support brace to decorate the support brace.

2. The support brace of claim 1, further comprising one or more decorative elements applied to an inside surface of the support brace, such that the support brace is reversible.

3. The support brace of claim 1, wherein the one or more decorative elements entirely cover all of a surface area of the support brace.

4. The support brace of claim 1, wherein a plurality of decorative elements are applied to the support brace.

5. The support brace of claim 4, wherein the plurality of decorative elements are applied in a repeating pattern.

6. The support brace of claim 1, wherein the support brace is a knee brace.

7. The support brace of claim 1, wherein the support brace is an elbow brace.

8. The support brace of claim 1, wherein the support brace is a wrist brace.

9. The support brace of claim 1, wherein the support brace is an ankle brace.

10. A decorated support bandage set comprising:
a support bandage strip;
one or more decorative elements applied to the support bandage strip over at least a portion of the support bandage strip; and
one or more decorated clips, each decorated clip comprising:
a top having a decorative element, and
a bottom having protruding teeth to engage a surface of the decorated bandage wrap to secure an end of the support bandage strip against itself after it is wrapped about a person.

11. The set of claim 10, wherein the one or more bandage decorative elements extend for an entire length of the support bandage strip.

12. The set of claim 10, wherein the support bandage strip includes at least cotton, silk, wool, or a synthetic fabric.

13. The set of claim 10, wherein the clip decorative element is applied to the decorated clip via an adhesive.

14. The set of claim 10, wherein the one or more decorative bandage elements match the clip decorative element of the one or more decorated clips.

15. The set of claim 10, further comprising additional decorative elements for a user to personalize the decorated bandage wrap or decorated clip.

16. The set of claim 10, wherein the one or more decorative elements are applied to the support bandage strip by stitching.

17. The set of claim 10, wherein the one or more decorative elements are applied to the support bandage strip by printing.

18. The set of claim 10, wherein the one or more decorative elements are applied to the support bandage strip by embroidery.

19. The set of claim 10, wherein the one or more decorative elements are applied to the support bandage strip by dyeing.

20. The set of claim 10, further comprising a support brace comprising a fabric material formed in a cylindrical shape to provide support to a body part, the fabric material defining a top opening and a bottom opening.
